(19) United States
(12) Patent Application Publication
(10) Pub. No.: US 2010/0184653 A1
(43) Pub. Date: Jul. 22, 2010

(54) Derivates of Polyethylene Glycol Modified Thymosin Alpha 1

(75) Inventor: Huijuan Zhong, Jiangsu Province (CN)

Correspondence Address: DORITY & MANNING, P.A., POST OFFICE BOX 1449, GREENVILLE, SC, 29602-1449, US

(73) Assignee: Jiangsu Hansen Pharmaceutical Co., Ltd., Lianyungang (CH)

(21) Appl. No.: 12/172,607

(22) Filed: Jul. 14, 2008

(51) Int. Cl. A61K 38/22; C07K 14/575; C07K 1/00

(52) U.S. Cl. 514/12; 530/324

(57) ABSTRACT

Pharmaceutical compositions that include thymosin alpha 1 peptide derivatives modified at the C-terminal of the peptide chain with polyethylene glycol, and their pharmaceutical acceptable salts, are generally disclosed. Also, new methods used to prepare these thymosin alpha 1 peptide derivatives modified at the C-terminal of the peptide chain with polyethylene glycol are generally provided. The presently disclosed compounds and their salts can be prepared administered to humans to treat immune disease and can also be used in adjuvant treatment.

### FIELD OF THE INVENTION

The present disclosure relates to Polyethylene Glycol (PEG) modified Thymosin alpha 1 with long-lasting effect, preparation methods and applications thereof.

### BACKGROUND OF THE INVENTION

The thymus is one of the important immune organs in the human body. The thymus plays a role in the development of lymphatic system, and the maintenance of normal function of immune system. The thymus also helps fight diseases through producing hormones that are beneficial to the anti-infection, anti-tumor, anti-autoimmune diseases and organ transplant functions. The thymus degrades as the person's age increase, and as a result the risk of tumor, infection and immune diseases rises gradually with age.

Recently, several thymus peptides have been isolated and purified, wherein the biological activity of thymosin alpha 1 (“TA1”) is found to be as 10-1000 times stronger as the mixture of thymus peptides. Thymosin alpha 1 is the fifth ingredient of thymus peptides, and is active in adjusting immunity, motivating sensitized cells to generate lymphokines like α-IFN, γ-INF and IL-2, strengthening IL-2 expression of cytokines, adjusting the terminal deoxynucleotidyl transferase (TdT) activity of lymphokines and spleen cells, building up the expression of Thy1 and Lyt 1, 2 and 3 of bone marrow precursor cells, accelerating NK cell generation, boosting NK cell activity, advancing mixed lymphocyte reaction by strengthening activity of adjuvant T-cell, and antagonizing thymus cell apoptosis during its grow. Testing in vivo shows that Thymosin alpha 1 also plays a role in the growth and function reorganization of T cells, and boosting IL-2 expression and IL-2 secretion by lymphocyte, strengthen host's resistance to infection, boost virus cleanness, strengthen immunity, resist oxidation and inhibit cancer cell growth. Thymosin alpha 1 has been used clinically for treatment of tumors, and in adjuvant therapy of anti-virus and immunodeficiency as well as used as vaccine enhancer. For example, Chien, et al. (Chien, R-N Et al, Hepatology, 1998; 27: 1383-138) applied thymosin alpha 1 to treat Chronic Hepatitis B. Their results showed that HBV DNA serum clearance and HBeAg negative turning rate were higher than the control group without using thymosin alpha 1, and according to Pathological examination, inflammation and fiber generation in therapy groups were far weaker than that in control groups.

Thymosin alpha 1 also has special effect when used to treat liver fibrosis. Interferon (IFN) is currently the only therapeutical reagent that has certain effect on liver fibrosis, whose side effects include such as low response rates and dose-limiting. In a randomized, placebo-controlled double-blind trial of combination therapy of thymosin alpha 1 and IFN for the treatment of chronic hepatitis C infection, Sherman, et al. (Sherman, K. E., et al. Hepatology, 1998; 27: 1128-35) observed that various detection index obtained from the treating groups were better than the counterpart obtained from control groups. For example, HCV RNA clearance was 37.1%, apparently being better than 16.2% of IFN groups. Thymosin alpha 1 can also be used to treat liver cancer. Stefanini, et al. (Stefanini G. F. et al. Hepatol-Gastroenterology, 1998; 45: 209-15) used the same to treat more than 12 liver cancer patients whose survival periods were prominently prolonged.

Thymosin alpha 1 is commercially available under trademark of Zadaxin (SciClone Pharmaceuticals, Inc., Foster City, Calif.) in the market, which is obtained by synthesis without containing other serum ingredient like albumin, etc. It is used to treat Hepatitis B, Hepatitis C, HIV infection and some kind of tumor such as melanoma, lung cancer, leukemia, P-cell carcinoma, colon cancer, and so on, with good effect. But the pharmacokinetics of thymosin alpha 1 is poor, wherein life time in body is short and T_{1/2} is only 2 hours. It has been proved in the animal model that thymosin alpha 1 can not be fully exert its activity in body, and frequent injection is needed, which results in, whereas, an inconvenient and expensive treatment. Moreover, treatment quality declines because many patients can not afford the expensive price of treatment.

Research shows that thymosin alpha 1 in bodies is derived from pro-thymosin alpha 1, which has the same biological activity as thymosin alpha 1 but has a different molecular weight. Pro-thymosin alpha 1 has longer in vivo biological activity because its larger molecular structure needs a longer time to be digested. So, one way to solve the problems mentioned above is to develop long effect derivative of thymosin alpha 1. Chemical modification of thymosin alpha 1 may be able to prolong its life time and improve its stability in bodies, which thus will bring down treatment cost and improve treatment quality.

In view of the successful protein modification with PEG, the same technology may be applied to modify thymosin alpha 1 in order to improve its pharmacokinetics, and furthermore to enhance the effect. WO 03/037272, which is incorporated by reference herein, discloses a method of modifying thymosin alpha 1 with polymers and use thereof. For example, modification of thymosin alpha 1 can be carried out through conjugating one or more of the five amino groups on thymosin alpha 1 with PEG having molecular weight of about 2000. Since thymosin alpha 1 has five amino groups (one in the N-terminal amino, the other four are side chain amino groups of lysines), and moreover, the modification is based on the five amino groups, selective modification can only be achieved by complex chemical synthesis in combination with strategy of group protection, which makes it very expensive in large scale manufacture and very difficult to commercialize. There is a need of developing method for preparing modified thymosin alpha 1 derivative that is suitable for industrial application.

Based on the above observation and comprehensive research in combination with specific chemical modification methods, the biological activity of thymosin alpha 1 could be retained as same as possible if the C-terminal of thymosin alpha 1 is modified with PEG. However, thymosin alpha 1 prepared by chemical synthesis or DNA recombinant strategy is difficult to be industrialized because of low yield. Therefore, only the derivative of thymosin alpha 1 (instead of thymosin alpha 1 itself) that can be manufactured easier should be used as a modification precursor. In other words, adding specific designed sequences and connections to the C-terminal of thymosin alpha 1 makes derivatives of thymosin alpha 1 easy to be prepared, and the polyethylene glycol modified derivatives of thymosin alpha 1 possess better biological activity. The precursor used to modification can be prepared by any known method in this art, chemical synthesis or DNA recombinant strategy is preferred.

Polyethylene glycol (PEG) modified derivatives of thymosin alpha 1 can be prepared according the method recorded in Chinese patent application No.200410037523. However, the present disclosure provides a different method in which the thymosin alpha 1 can be modified with new amino acid residues. The present method has not only good yield, but also the biological activity of the obtained derivatives are better than the compounds prepared with method disclosed in Chinese patent application No. 200410037523. X.

### SUMMARY OF THE INVENTION

Objects and advantages of the invention will be set forth in part in the following description, or may be obvious from the description, or may be learned through practice of the invention.

In one embodiment, the present disclosure is directed to pharmaceutical compositions including a thymosin alpha 1 peptide derivative modified at the C-terminal of the peptide chain with polyethylene glycol and their pharmaceutical acceptable salts. In another embodiment, the present disclosure provides new methods used to prepare these compounds. The presently disclosed compounds and their salts can be administered to humans to treat immune disease and can also be used in adjuvant treatment.

Other features and aspects of the present invention are discussed in greater detail below.

### DETAILED DESCRIPTION OF THE FIGURES

A full and enabling disclosure of the present invention, including the best mode thereof to one skilled in the art, is set forth more particularly in the remainder of the specification, which includes reference to the accompanying figures, in which:

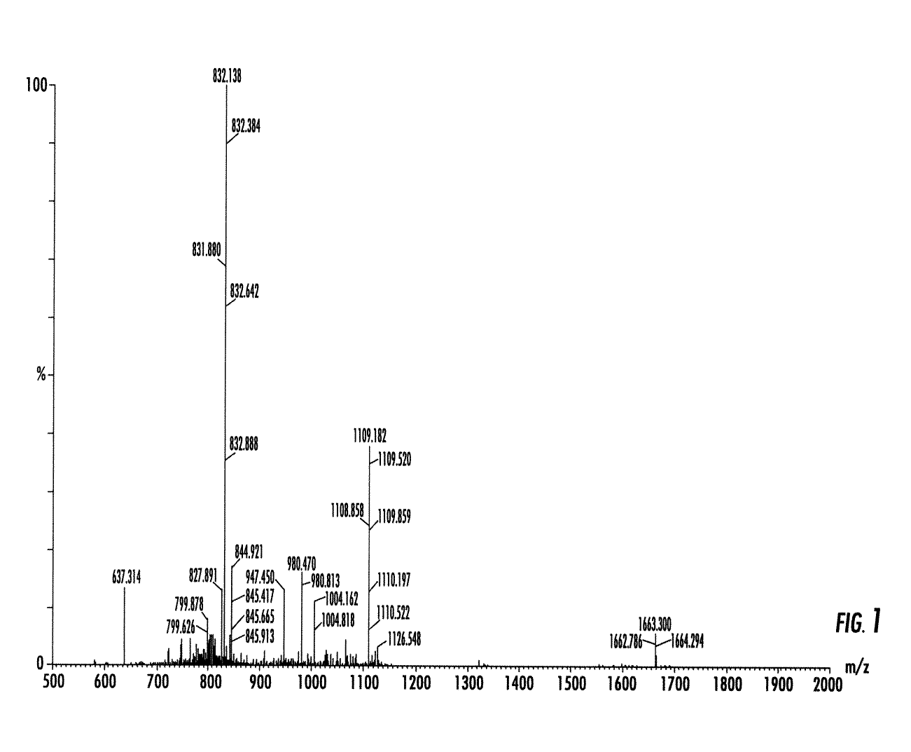

FIG. 1 indicates MALDI-TOF spectrum of thymosin alpha 1 derivative obtained in example 1;

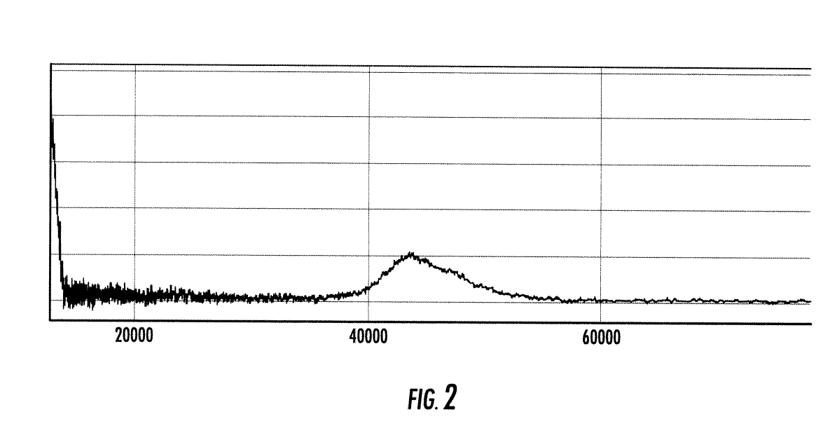

FIG. 2 indicates MALDI-TOF spectrum of thymosin alpha 1 derivative modified with PEG obtained in example 1;

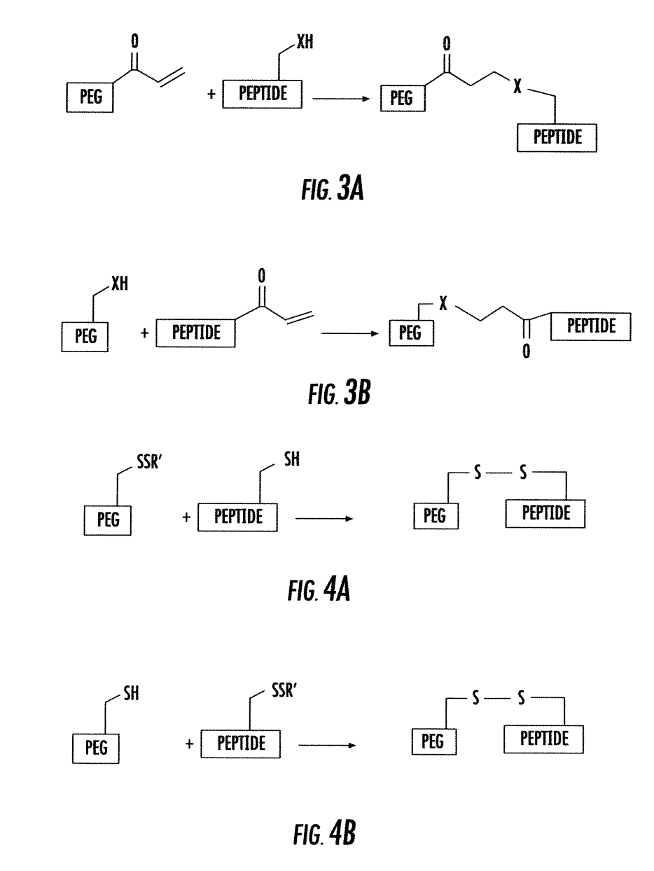

FIG. 3(a) shows an exemplary preparation method of thymosin alpha 1 derivatives modified with PEG and their pharmaceutical acceptable salts by reacting a PEG containing Michael Addition receptor and thymosin alpha 1 derivatives containing Michael Addition donator (such as homocysteine);

FIG. 3(b) shows an exemplary preparation method of thymosin alpha 1 derivatives modified with PEG and their pharmaceutical acceptable salts by reacting a PEG containing Michael Addition donator and thymosin alpha 1 derivatives containing Michael Addition receptor;

FIGS. 4(a) and 4(b) show reaction between PEG which can generate asymmetry disulfide bond and thymosin alpha 1 derivatives;

FIGS. 5(a) and 5(b) show reaction between PEG derivatives containing activated carboxyl and thymosin alpha 1 derivatives whose C-terminal is lysine or histidine, respectfully;

FIG. 6 shows a reductive amination reaction between PEG derivatives containing carbonyl group and thymosin alpha 1 derivatives whose C-terminal is lysine;

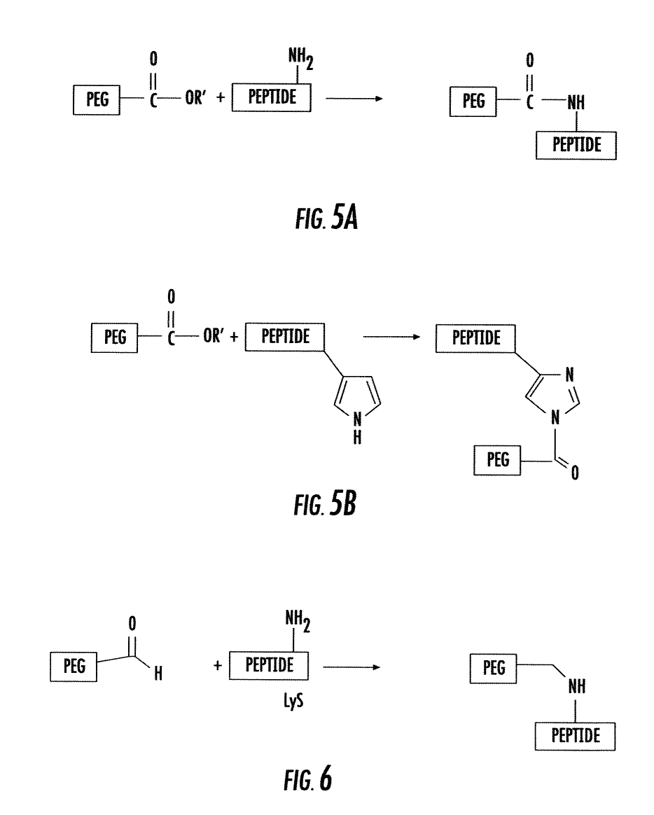

FIG. 7 shows a reaction between PEG derivatives containing isocyano or isothiocyano group and thymosin alpha 1 derivatives whose C-terminal is lysine;

FIG. 8 shows a reaction between PEG derivatives containing activated carbonyl group and thymosin alpha 1 derivatives whose C-terminal is lysine or histidine;

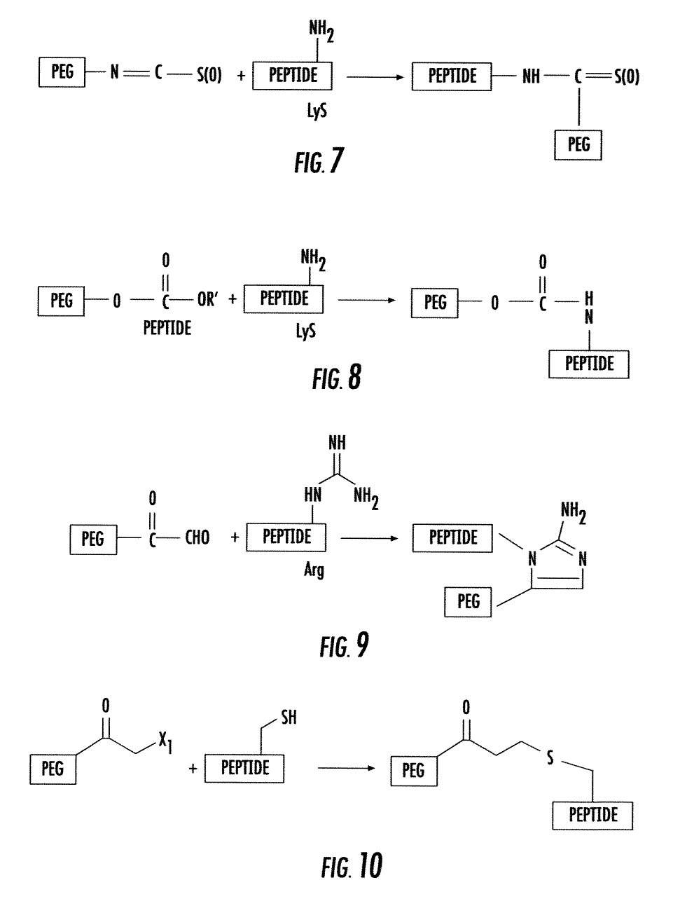

FIG. 9 shows a reaction between PEG derivatives containing 2-carbonyl acetaldehyde group and thymosin alpha 1 derivatives whose C-terminal is argine; and

FIG. 10 shows a nucleophilic substitution reaction between PEG derivatives containing easy leaving groups and thymosin alpha 1 derivatives containing sulphydryal group, wherein, X₁ in method (7) represents halogen or sulfonate.

### DETAILED DESCRIPTION OF THE INVENTION

Reference now will be made to the embodiments of the invention, one or more examples of which are set forth below. Each example is provided by way of an explanation of the invention, not as a limitation of the invention. In fact, it will be apparent to those skilled in the art that various modifications and variations can be made in the invention without departing from the scope or spirit of the invention. For instance, features illustrated or described as one embodiment can be used on another embodiment to yield still a further embodiment. Thus, it is intended that the present invention cover such modifications and variations as come within the scope of the appended claims and their equivalents. It is to be understood by one of ordinary skill in the art that the present discussion is a description of exemplary embodiments only, and is not intended as limiting the broader aspects of the present invention, which broader aspects are embodied exemplary constructions.

In one embodiment, the present disclosure is generally directed to thymosin alpha 1 peptide derivatives modified with PEG, and their pharmaceutical acceptable salts. The characteristics of these molecules are that the C-terminal of the thymosin alpha 1 peptide derivatives is bonded to PEG. The molecular weight of the PEG can be within a range of 5,000-80,000, preferably within a range of 8,000-60,000, more preferably within a range of 10,000-50,000, most preferably within a range of 20,000-40,000. The PEG may be straight-chain or branched-chain, preferably branched-chain.

In one particular embodiment, the thymosin alpha 1 peptide derivatives modified with PEG, and their pharmaceutical acceptable salts, have the following formula (1):

| | |
|---|---|
| (1): | A-Ser-Asp-Ala-Ala-Val-Asp-Thr-Ser-Ser-Glu- |
| | Ile-Thr-Thr-B-Asp-Leu-C-Glu-D-E-Glu-Val-Val- |
| | Glu-Glu-Ala-Glu-Asn-X-Y-Z |

wherein,
A represents H or Ac;
B, C, D, E represent Lys or Arg;
X is selected from the group consisting of (Gly)ₙ, (Gly-Ser)ₙ, (Gly-Gly-Ser)ₙ and (Ser-Gly-Gly)ₙ, where 1≦n≦10;
Y is connection site selected from the group consisting of Cys, homoCys, Lys, Arg and His, and Y is a residue modified with PEG; and
Z represents OH or NH₂.

The thymosin alpha 1 peptide derivatives in the present invention have the following formula (2):

| | |
|---|---|
| (2) | A-Ser-Asp-Ala-Ala-Val-Asp-Thr-Ser-Ser-Glu-Ile- |
| | Thr-Thr-B-Asp-Leu-C-Glu-D-E-Glu-Val-Val-Glu- |
| | Glu-Ala-Glu-Asn-X |

wherein,
A represents H or Ac;
B, C, D, E represent Lys or Arg;
X is selected from the group consisting of (Gly)ₙ, (Gly-Ser)ₙ, (Gly-Gly-Ser)ₙ and (Ser-Gly-Gly)ₙ, where 1≦n≦10;

The thymosin alpha 1 peptide derivatives are selected from sequences mentioned in SEQ ID No.1-24, preferably sequences mentioned in SEQ ID No.1-12, in the attached sequence listing.

In a preferred embodiment, the thymosin alpha 1 peptide derivatives modified with PEG, and their pharmaceutical acceptable salts, have the following formula (3):

| | |
|---|---|
| (3): | A-Ser-Asp-Ala-Ala-Val-Asp-Thr-Ser-Ser-Glu- |
| | Ile-Thr-Thr-B-Asp-Leu-C-Glu-D-E-Glu-Val-Val- |
| | Glu-Glu-Ala-Glu-Asn-X-Y-Z |

wherein,
A represents Ac;
B, C, D, E each represent Lys;
X represents Gly-Gly;
Y represents Cys modified with PEG4K
   or
A represents Ac;
B, C, D, E represent Arg;
X represents Gly-Gly;
Y represents Cys modified with PEG4K.

The present disclosure also provides preparation methods of the thymosin alpha 1 peptide derivatives modified with PEG and their pharmaceutical acceptable salts. The method comprises the steps of:
(1) Preparing (a) a PEG containing Michael Addition receptor and thymosin alpha 1 peptide derivatives containing Michael Addition donator (such as homocysteine); or (b) preparing a PEG containing Michael Addition donator and thymosin alpha 1 peptide derivatives containing Michael Addition receptor; and
(2) Reacting the PEG containing Michael Addition donator or receptor with thymosin alpha 1 peptide derivatives to produce thymosin alpha 1 peptide derivatives modified with PEG.

These alternative methods are shown in FIGS. 3(a) and 3(b), respectfully, wherein X represents N or S, the double bond group is from H, C₁₋₄ alkyl, C₃₋₆ heterocyclic or aromatic heterocyclic group.

In these methods, the cysteine, homocysteine, lysine, histidine on the C-terminal of thymosin alpha 1 peptide derivatives are the point for PEG modification. In other words, the C-terminal of thymosin alpha 1 peptide derivatives reacts with the polyethylene glycol on the residue of cysteine, homocysteine, lysine or histidine on the C-terminal.

The following methods can be chosen as specific chemical modification reaction to form covalent bond:
(1) Reaction between PEG which can generate asymmetry disulfide bond and thymosin alpha 1 peptide derivatives, as shown in FIGS. 4(a) and 4(b).
(2) Reaction between PEG derivatives containing activated carboxyl and thymosin alpha 1 peptide derivatives whose C-terminal is lysine or histidine, as shown in FIGS. 5(a) and 5(b).
(3) Reductive Amination reaction between PEG derivatives containing carbonyl group and thymosin alpha 1 peptide derivatives whose C-terminal is lysine, as shown in FIG. 6.
(4) Reaction between PEG derivatives containing isocyano or isothiocyano group and thymosin alpha 1 peptide derivatives whose C-terminal is lysine, as shown in FIG. 7.
(5) Reaction between PEG derivatives containing activated carbonyl group and thymosin alpha 1 peptide derivatives whose C-terminal is lysine or histidine, as shown in FIG. 8.
(6) Reaction between PEG derivatives containing 2-carbonyl acetaldehyde group and thymosin alpha 1 peptide derivatives whose C-terminal is argine, as shown in FIG. 9.
(7) Nucleophilic Substitution reaction between PEG derivatives containing easy leaving groups and thymosin alpha 1 peptide derivatives containing sulphydryal group, as shown in FIG. 10, wherein, X₁ in method (7) represents halogen or sulfonate.

In other words, present disclosure provides thymosin alpha 1 peptide derivatives modified with PEG and their pharmaceutical acceptable salts, where the C-terminal of thymosin alpha 1 peptide derivatives is modified with PEG. The preferred structures are shown in formula (4) as follows:

| | |
|---|---|
| (4): | A-Ser-Asp-Ala-Ala-Val-Asp-Thr-Ser-Ser-Glu- |
| | 15 20 |
| | Ile-Thr-Thr-B-Asp-Leu-C-Glu-D-E-Glu-Val-Val- |
| | 25 30 |
| | Glu-Glu-Ala-Glu-Asn-X-Y-Z |

wherein,
A represents H or Ac;
B, C, D, E present Lys or Arg,
X is selected from the group consisting of (Gly)ₙ, (Gly-Ser)ₙ, (Gly-Gly-Ser)ₙ and (Ser-Gly-Gly)ₙ, where 1≦n≦10;
Y is connection point, which is selected from the group consisting of Cys, homoCys, Lys, Arg and His, and Y is residue modified with PEG;
Z is OH or NH₂; and
if Y is Cys or homoCys, PEG is conjuncted with thymosin alpha 1 peptide derivatives by S—C or S—S bonds; if Y is Lys, PEG is conjuncted with thymosin alpha 1 peptide derivatives by carboxamide bond or secondary amino bonds; if Y is His, PEG is conjuncted with thymosin alpha 1 peptide derivatives by forming acylimidazole with nitrogen atom in the imidazole cycle of Histidine; if Y is Arg, PEG is conjuncted with thymosin alpha 1 peptide derivatives by forming heterocycle.

The preferred amino acids or groups are defined as follows:
A is Ac, B, C, D, E are Lys respectively, X is Gly-Gly, Y is Cys modified with PEG40K, or
A is Ac, B, C, D, E are Arg respectively, X is Gly-Gly, Y is Lys modified with PEG40K.

The C-terminal of thymosin alpha 1 peptide derivatives in the invention can be coupled on one or both ends of the PEG, preferred to be coupled on one end.

The thymosin alpha 1 peptide derivatives modified with PEG in this disclosure are amphoteric compounds, which can be prepared into salted with general acids or bases in this art. The acids could be selected from the group consisting of hydrochloric acid, hydrobromic acid, iodic acid, carbonic acid, phosphoric acid, p-toluenesulfonic acid, methanesulfonic acid, p-bromophenyl carbonic acid, carbonic acid, succinic acid, citric acid, benzoic acid, acetic acid, fluoroacetic acid, et al. Examples of the salts include sulfate, pyrosulfate, disulfate , sulfite, bisulfite, triphosphate, biphosphate salts, phosphate salts, metaphosphate, pyrophosphate, hydrochloride, bromide, iodide, acetate, trifluoro acetate, propionate, caprate, caprylate, acrylate, formate, isobutyrate, caproate, heptanate, propiolate, benzoate, malonate, succinate salt, suberate, decanedioate, fumaric acid salt, maleic acid salt 2-butyne-1,4-diate, hexyl-1,6-diate, benzoate, chlorobenzoate, methyl-benzoate, dinitrobenzate, hydroxybenzate, methoxy-benzoate, phenylcaproate, phenylpropionate, phenylbutyrate, citrate, lactate, r-hydroxybutyrate, hydroxylacetate, tartrate, methyl-sulfonate, propanyl-sulfonate, naphthalene-1-sulfonate, naphthalene-2-sulfonate, mandelate, hydrochloric acid, hydrobromic acid, acetic acid. Trifluoroacetic acid is preferred, and acetic acid is more preferred.

The base could be ammonium, hydroxides of alkali metals or of alkaline earth, carbonate, and bicarbonate, sodium hydroxide, potassium hydroxide, ammonium hydroxide, sodium carbonate. Potassium carbonate are preferred.

The preparation of thymosin alpha 1 peptide derivatives in this invention can be conducted according to the general methods in this art. The preferred methods are solid-phase and liquid-phase chemical synthesis, wherein solid-phase synthesis includes protecting amino group with Fmoc or Boc, synthesizing amino acid sequences through automated or manual peptide synthesis, then cleaving, purifying through high performance liquid chromatograph (HPLC), lyophilizing, the obtained polypeptides are the precursor for pegylation. The thymosin alpha 1 peptide derivatives in the invention are selected from SEQ ID NO: 1-24.

The thymosin alpha 1 peptide derivative in this invention can also be prepared through genetic engineering; by the following steps of:
a) Synthesizing the gene fragment in term of the amino acid sequence of the thymosin alpha 1 peptide derivative;
b) Obtaining strains after gene fragment ligation, plasmid construction, solid culture, transformation and clone;
c) Extracting inclusion body from the strains which is fermented and cell disrupted;
d) Obtaining crude product following lysis of inclusion body, the crude product is further isolated and purified by HPLC and then lyophilized to obtain the precursor to be modified with PEG.

The preparation method of thymosin alpha 1 peptide derivative modified with PEG in present invention can, in one embodiment, comprises the steps of:

1) Preparing or obtaining
(a) PEG containing Michael Addition receptor and thymosin alpha 1 peptide derivatives containing Michael Addition donator such as cysteine; or
(b) PEG containing Michael Addition donator and thymosin alpha 1 peptide derivatives containing Michael Addition receptor;
2) Reacting the PEG and the thymosin alpha 1 peptide derivatives containing Michael Addition donators or receptors, respectfully, to prepare thymosin alpha 1 peptide derivatives modified with PEG, for example, making maleimide derived PEG react with thymosin alpha 1 peptide derivatives containing cysteine.

The following methods can be chosen to prepare thymosin alpha 1 peptide derivative modified with PEG in present invention,
(1) Reaction between PEG which can generate asymmetry Disulfide bond and thymosin alpha 1 peptide derivatives. For example, the reaction is made between PEG containing activated disulfide bond and thymosin alpha 1 peptide derivatives containing cysteine to obtain product;
(2) Reaction between PEG derivatives containing activated carboxyl and thymosin alpha 1 peptide derivatives whose C-terminal is lysine or histidine to obtain product conjuncted by amide bond, wherein, the four lysine in the structure of thymosin alpha 1 derivative are substituted by arginine;
(3) Reductive Amination reaction between PEG derivatives containing carbonyl group and thymosin alpha 1 derivatives with C-terminal being lysine to obtain product, wherein, the four lysine in the structure of thymosin alpha 1 peptide derivative are substituted by arginines;
(4) Amino addition reaction between PEG derivatives containing isocyano or isothiocyano group and thymosin alpha 1 peptide derivatives whose C-terminal is lysine to obtain the product, wherein, the four lysine in the structure of thymosin alpha 1 peptide derivative are substituted by arginines;
(5) Reaction between PEG derivatives containing 2-carbonyl acetaldehyde group and thymosin alpha 1 peptide derivatives whose C-terminal is arginine to obtain product;
(6) Nucleophilic Substitution reaction between PEG derivatives containing easy leaving groups (such as I, Br, Cl) and thymosin alpha 1 peptide derivatives containing sulphydryal group.

The compounds in of the present disclosure include thymosin alpha 1 peptide derivatives modified with PEG as well as the new intermediates prepared during the process. All of these compounds can be used to reorganize immune function for immune-lost or immune compromised patients, and can be used to treat immune deficiency diseases, such as influenza, chronic hepatitis, immune compromised and tumor or virus infection diseases, especially be used as adjuvant agent to treat tumor or virus infection.

It has been proved that thymosin alpha 1 peptide derivatives modified with PEG have the same effect as natural thymosin alpha 1. Also, pharmacokinetic test shows that half life of thymosin alpha 1 peptide derivatives modified with PEG administrated by hypodermic injection in this invention is 2-3 days in vivo, but only about 2 hours for the natural thymosin alpha 1 administrated in the same way.

The dosage of the thymosin alpha 1 peptide derivatives modified with PEG of the present disclosure as an adjuvant agent for enhancing immune can be determined through routing dosage titration, which is 1-100 mg/Kg body weight a week, preferred 5-50 mg/Kg body weight a week, more preferred 20-50 mg/Kg body weight a week. The thymosin alpha 1 peptide derivatives modified with PEG can be prepared into injection for use with pharmaceutical liquid like water.

The thymosin alpha 1 peptide derivatives modified on the C-terminal with PEG of the present disclosure are prepared easier than the thymosin alpha 1 peptide derivatives modified on N-terminal with PEG. Additionally, the problems associated with the modification of N-terminal with PEG, such as it being difficult to upscale to large scale in industry and the product being difficult to obtain, can be overcome by presently disclosed methods. Moreover, the obtained compounds of the present disclosure possess better activity.

The invention is further illustrated by the following examples, but is not limited to the illustrations.

### Examples

### Example 1

### Preparation of

Ac-Ser-Asp-Ala-Ala-Val-Asp-Thr-Ser-Ser-Glu-Ile-
Thr-Thr-Lys-Asp-Leu-Lys-Glu-Lys-Lys-Glu-Val-Val-
Glu-Glu-Ala-Glu-Asn-Gly-Gly-Cys-PEG40K

Step 1: Preparation of Precursor to be Modified with PEG-C-cys Thymosin Alpha 1 Peptide Derivative, Through Solid Phase Synthesis

### (1). Amino Acid Monomers Applied

Fmoc-Lys(Boc)-OH
Fmoc-Asn(Trt)-OH Fmoc-Ser(tBu)-OH
Fmoc-Asp(OtBu)-OH Fmoc-Thr(tBu)-OH
Fmoc-Cys(Trt)-OH Fmoc-Val-OH
Fmoc-Glu(OtBu)-OH Fmoc-Ile-OH
Fmoc-Gly-OH
   wherein, the abbreviations represent respectively:
Fmoc: 9-fluorenylmethoxycarbonyl
Boc: tert-butyloxycarbonyl
Trt: trityl
OtBu: tert-butyloxy
tBu: tert-butyl
PEG40K: branched-chain (mPEG)₂-MAL, molecular weight of mPEG is 20K

### (2) Instruments and Reagents

Instruments: the peptide sequence is synthesized through manual peptide synthesis, and the vessels are specific custom-made.

### Reagents:

N,N-dimethylformide (DMF),
dichloromethane (DCM),
piperidine, isopropanol, methanol,
N,N-Diisopropylcarbodimide
1-hydroxybenzotriazole (HOBt)

### (3) Operation:

10 g (6 mmol) Wang resin (0.6 mmol/g) coupled with Fmoc-Cys(Trt)-OH (Fmoc-Cys(Trt)-OH-Wang resin) was placed in a manual peptide synthesis vessel with a sintered glass filter on its bottom and a mechanical stirrer on the top. After being washed with 150 ml DMF, the resin was treated with 150 ml (divided into two portions) 20% of the piperidine in DMF twice for 10 minutes each to removal Fmoc protection on amino group, then it was washed 3 times with 150 ml DMF each (450 ml together), filtered. The deprotected amino resin reacted with 8.95 g (15 mmol) Fmoc-Gly-OH in 150 ml DMF, 2.3 g (15 mmol) 1-hydroxybenzotriazole, and 2.34 ml (15 mmol) N,N-Diisopropylcarbodimide for two hours to obtain Fmoc-Gly-Cys(Trt) Wang Resin, the result of Ninhydfin Color Test was negative. After that, the solid phase synthesis was carried out in according with the flowing steps, wherein, each amino acid was attached in sequence to the enlonging peptide chain (otherwise stated), the resin was washed with 20 volume solvent or solution for each time. Otherwise specific statement, all amino acid derivatives were L configuration.
(1) The resin was washed with 20% piperidine in DMF;
(2) The washed resin was stirred in 20% piperidine in DMF for 30 min;
(3) Then it was washed for 3 times with DMF;
(4) The resin was mixed with 15 mmol Fmoc-Gly-OH, 1-hydroxybenzotriazole, and N,N-Diisopropylcarbodimide respectively in DMF, and the obtained mixture was stirred for 120 min;
(5) The obtained product was washed 2 times with isopropanol;
(6) It was washed 3 times with DMF; and
(7) If the result of Ninhydfin Color Test was positive, it was necessary to repeat steps 4-6; if it was negative, the next reaction cycle was carried out.

The following Fmoc-amino acids were used, and in each steps (4 and 7), the synthesis cycle was repeated by applying corresponding amino acid.
Fmoc-Asn(Trt)-OH, Fmoc-Glu(OtBu)-OH, Fmoc-Ala-OH,
Fmoc-Glu(OtBu)-OH Fmoc-Glu(OtBu)-OH Fmoc-Val-OH,
Fmoc-Val-OH, Fmoc-Glu(OtBu),
Fmoc-Lys(Boc)-OH, Fmoc-Lys(Boc)-OH, Fmoc-Glu(OtBu)-OH,
Fmoc-Lys(Boc)-OH, Fmoc-Leu-OH, Fmoc-Asp(OtBu)-OH,
Fmoc-Lys(Boc)-OH, Fmoc-Thr(tBu)-OH, Fmoc-Thr(tBu)-OH,
Fmoc-Ile-OH, Fmoc-Glu(OtBu)-OH, Fmoc-Ser(tBu)-OH,
Fmoc-Ser(tBu)-OH, Fmoc-Thr(tBu)-OH, Fmoc-Asp(OtBu)-OH,
Fmoc-Val-OH, Fmoc-Ala-OH, Fmoc-Ala-OH,
Fmoc-Asp(OtBu)-OH, Fmoc-Ser(tBu)-OH,

Last, the synthesis cycle was completed by acetylating with acetic anhydride and thymosin alpha 1-Cys(Trt)-Wang Resin was thus obtained.

### Structure:

Ac-Ser((tBu)-Asp(OtBu)-Ala-Ala-Val-Asp(OtBu)-
Thr(tBu)-Ser(tBu)-Glu(OtBu)-Ile-Thr(tBu)-Thr(tBu)-
Lys(Boc)-Asp(OtBu)-Leu-Lys(Boc)-Glu(OtBu)-
Lys(Boc)-Lys(Boc)-Glu(OtBu)-Val-Val-Glu(OtBu)-
Glu(OtBu)-Ala-Glu(OtBu)-Asn(Trt)-Gly-Gly-Cys(Trt)-
Wang resin

Weight: 42 g

5.0 g obtained thymosin alpha 1 derivatives-Cys(Trt)-Wang Resin was stirred with 50 ml trifluoroacetic acid (TFA) containing 2% thioanisole, 2% methanol, 4% triisopropylsilane, 2% ethanedithiol 2hours. After TFA being removed and ether precipitation, the obtained crude product was dissolved in 100 ml 0.2M ammonium acetate buffer. After being filtered, the obtained filtrate was separated and purified through preparative HPLC.

### Preparative HPLC Condition:

Flow rate: 10 ml/min UV: 234 nm
Column: Vydac, C18 (2.2×25 cm, 10 u)
Gradient: 0.5% acetonitrile/min (buffer A: 0.01% TFA water solution; buffer B: 0.01% TFA-acetonitrile solution;),

After purification through HPLC, the fraction containing product was combined, which was lyophilized and 0.6 g product was obtained. Molecular weight determined by MS was right: (M+4)⁺⁴=832, (M+3)⁺³=1109, (M+2H)⁺²=1663. (calculated molecular weight=3324, see FIG. 1).

Step 2: Preparation of Thymosin Alpha 1 Derivative Modified with PEG Through Reaction Between Thymosin Alpha 1 Derivative and PEG

50 mg of C-terminal cysteine substituted thymosin alpha 1 derivative and 500 mg of methoxy-PEG modified with maleimide (molecular weight of 40000 Dalton) were dissolved in 5 ml 0.1M phosphate buffer solution and reacted for two hours, after being desalted with Superdex, the product was obtained by inverse preparative HPLC, which was then lyophilized, yielded 520 mg title product, #: T-GG-C-mPEG. [MALDI-TOF mass spectrometry showed that the molecular weight was:
40000-46000, see FIG. 2]

### Preparative HPLC Condition:

Flow rate: 10 ml/min; UV: 234 nm
Column: Vydac, C18 (2.2×25 cm, 10 u)
Gradient: 0.5% acetonitrile/min (0.01% TFA water solution; 0.01% TFA acetonitrile solution;).

### Example 2

### Preparation of

Ac-Ser-Asp-Ala-Ala-Val-Asp-Thr-Ser-Ser-Glu-Ile-
Thr-Thr-Arg-Asp-Leu-Arg-Glu-Arg-Arg-Glu-Val-Val-
Glu-Glu-Ala-Glu-Asn-Gly-Gly-Lys-PEG40K

Step 1: Preparation of C-Lys Thymosin Alpha 1 Derivative Whose C-14,17,19,20 were Substituted by Arginine, as Precursor Used to be Modified with PEG, Through Solid Phase Synthesis Method

Method applied was similar to that described in example 1, the difference was that 10 g (6 mmol) Wang resin (0.6 mmol/g) coupled with Fmoc-Lys(Boc)OH was used to couple with the following Fmoc-amino acid, wherein synthesis cycle was repeated by using corresponding amino acid in each coupling cycle:
Fmoc-Gly-OH, Fmoc-Gly-OH, Fmoc-Asn(Trt)-OH
Fmoc-Glu(OtBu)-OH, Fmoc-Ala-OH, Fmoc-Glu(OtBu)-OH,
Fmoc-Glu(OtBu)-OH, Fmoc-Val-OH, Fmoc-Val-OH,
Fmoc-Glu(OtBu)-OH, Fmoc-Arg(Pbf)-OH, Fmoc-Arg(Pbf)-OH,
Fmoc-Glu(OtBu)-OH, Fmoc-Arg(Pbf)-OH, Fmoc-Leu-OH,
Fmoc-Asp(OtBu)-OH, Fmoc-Arg(Pbf)-OH, Fmoc-Thr(tBu)-OH,
Fmoc-Thr(tBu)-OH, Fmoc-Ile-OH, Fmoc-Glu(OtBu)-OH,
Fmoc-Ser(tBu)-OH, Fmoc-Ser(tBu)-OH, Fmoc-Thr(tBu)-OH,
Fmoc-Asp(OtBu)-OH, Fmoc-Val-OH, Fmoc-Ala-OH,
Fmoc-Ala-OH, Fmoc-Asp(OtBu)-OH, Fmoc-Ser(tBu)-OH,

Last, the cycle was completed by acetylating with acetic anhydride. The title peptide on resin was thus obtained.

Methods of cleaving the peptide from resin and separating and purifying the crude product were similar to that described in example 1.

Step 2 Preparation of Thymosin Alpha 1 Derivative Modified with PEG Through Reaction of Thymosin Alpha 1 Derivative and PEG

50 mg of C-terminal Lysine substituted thymosin alpha 1 derivative prepared in step 1 was dissolved in 5 ml 0.1M sodium bicarbonate solution and then 550 mg (molecular weight of 40000 Dalton) of carboxyl containing PEG activated with N-hydroxysuccinimide was added, pH was controlled at 8-9, reaction time was 4 hours. After being desalted with Superdex, the product was obtained by reverse preparative HPLC, lyophilized, 490 mg.

### Condition Applied in Reverse Preparative HPLC:

Flow rate: 10 ml/min; UV: 234 nm; Column: Vydac, C18 (2.2×25 cm, 10 u);
Linear Gradient: 0.5% acetonitrile/min (0.01% trifluoroacetic acid (TFA) in water solution; 0.01% TFA in acetonitrile solution;).

### Trial 1

### Activity Test:

With Zadaxin as a positive control, the effect of the samples on T-lymphocytes producing cytokines were tested to confirm if the samples could adjust T-lymphocyte immune activity and the extent thereof.

### [Principle]

In the lymphocytes cultured in vitro, T cell can be activated by the stimulation of mitosis Concanavalin A and brings change of cells' behavior such as cytokine synthesis, cytokine receptor expression, cell differentiation and cell proliferation. The generation of cytokine and proliferation of the cell are response of the immune cell function and cell activation. Detection means is reliable, simple, and with good repeatability, therefore, evaluating the samples' effect to the function of T lymphocytes by detecting cytokine generation and cell proliferation has been widely applied.

### [Materials]

### 1. Samples:

Zadaxin, thymosin alpha 1 modified with PEG (# PEG-TA1, that was, the thymosin alpha 1 peptide derivative modified with PEG in this invention when n was 0) and thymosin alpha 1 peptide derivative modified with PEG(SEQ IDQNO 1 #T-GG-C-mPEG), all samples above were diluted with 1640 culture media according to the experimental requirement.

### 2. Animals:

ICR mice are applied, male, 6-8-week-old, available from Shanghai Experimental Animal Center of the Chinese Academy of Sciences, certification No: SCXK (Shanghai) 2002-0010, which had been grown for 3-4 days in clean animal-room before experiment.

### 3. Reagent:

ConA (Concanavalin A) was available from Sigma Corporation.

RPMI 1640 culture fluid was prepared from GIBCO, containing 10% non-active bovine serum, 10 mm HEPES buffer, 100 IU/mL penicillin, 100 g/ml streptomycin, 2 mM L-glutamine, and 50 μM α-mercaptoethanol, PH 7.2.

Cytokine IFN-r and IL-2 et al. ELISA kit (produced by Becton Dickinson co.)

### [Procedure]

Preparation of spleen cell suspension: mice's spleen was obtained under aseptic condition, which was ground with frosted glass. The spleen cell suspension was thus obtained. After red blood lysis, the spleen cell suspension was washed three times, counted (live cells being more than 95%). The concentration of spleen cell was adjusted to 5×10⁶ cells/ml with 10% FBS (bovine serum) RPMI1640 culture fluid.

### 1. Content Assay of IL-2 and IFN-γ (ELISA Method)

### Preparation of Culture Supernatant

The concentration of mice's Spleen cells was adjusted to 5×10⁶ cells/ml, and then distributed it into 24 wells plate with 1 ml/well; samples with various concentrations were added into the plate respectively with 0.5 ml/well; and then ConA (20 ug/ml) was added with 0.5 ml/well. The samples were kept at 37° C. in CO₂ incubator for 24 hours, centrifuged and collected culture supernatant.

### 2. ELISA Method:

Antibody Coating: The antibody was diluted with coating buffer and then was added into 96 well ELISA plates with 50 μL each wells. The plate was left overnight at 40° C.

Blocking: The antibody coating buffer was removed, and the plate was washed 3 times with washing solution (PBS/Tween solution). 100 μL of blocking buffer (PBS/10% FBS) was added to each wells of the plates. The plate was incubated at room temperature for 1 hour.

Standards and samples: The blocking buffer was removed from the plates, which was washed 3 times with washing solution (PBS/Tween solution) and followed by adding standards and samples with 50 μL each wells. The plate was incubated at room temperature for 2 hours.

Detection of Antibody and Enzyme: The standards and samples were removed from the plates. The plate was washed 3 times with washing solution (PBS/Tween solution). The diluted detective antibody and enzyme HRP were added into the plate with 50 μL each wells. The plate was incubated at room temperature for 1 hour.

Substrate Staining: The diluted detection antibody and enzyme HRP were removed from the plate which was then washed 3 times with washing solution (PBS/Tween solution). The substrate resolved in citricic acid/hydrogen peroxide (TMB) was added into the plate with 50 μL each wells. The plate was incubated at room temperature away from the light for 30 minutes. After being stained, stop solution 2N H₂SO₄ was added into the plate with 25 μL per well.

OD Detection: The plate was placed into enzyme-labeled instrument and read OD values at 450 nm (calibrated value of 570 nm).

### [Result]

Determination of the effect of samples to ConA-induced cytokine production of T

### Lymphocytes:

| | Concentration | | | | |
|---|---|---|---|---|---|
| sample | (ug/ml) | Mean | SD | Mean | SD |
| control | | 4122 | 77 | 2920 | 24 |
| Zadaxin | 0.001 | 4177 | 177 | 3231 | 30 |
| | 0.01 | 4118 | 134 | 3582 | 25 |
| | 0.1 | 4106 | 191 | 3434 | 184 |
| | 1 | 4836 | 208 | 3692 | 19 |
| | 10 | 4832 | 36 | 3614 | 304 |
| | 100 | 4095 | 427 | 3840 | 476 |
| PEG-TA1 | 0.001 | 4809 | 151 | 3505 | 296 |
| | 0.01 | 4603 | 234 | 3438 | 117 |
| | 0.1 | 4141 | 200 | 3339 | 134 |
| | 1 | 4949 | 367 | 3214 | 55 |
| | 10 | 3874 | 3 | 3348 | 293 |
| | 100 | 5467 | 73 | 3582 | 25 |
| T-GG-C-mPE | 0.001 | 4703 | 163 | 3409 | 263 |
| | 0.01 | 4891 | 195 | 3610 | 125 |
| | 0.1 | 4654 | 203 | 3530 | 98 |
| | 1 | 4990 | 242 | 3397 | 72 |
| | 10 | 5102 | 103 | 3618 | 174 |
| | 100 | 5577 | 165 | 3692 | 184 |

### Conclusion:

In the experiments of ConA-induced cytokine production, Zadaxin showed a certain promoting activity to the production of IFN-r by T cells when its concentration was 1 ug/ml and 10 ug/ml; compared with Zadaxin, the thymosin alpha 1 peptide modified with PEG (PEG-TA1) with several experimental concentration was more effective to stimulate the production of IFN-r by T cells. However, the thymosin alpha 1 peptide derivative modified with PEG (SEQ ID NO 1, # T-GG-C-mPEG) with all experimental concentrations showed activity of promoting the production of IFN-r by T cells, and moreover, it is more effective than the thymosin alpha 1 modified with PEG at the N-terminal (PEG-TA1).

What is claimed is:

1. A pharmaceutical composition comprising a Thymosin alpha 1 peptide derivative, wherein the thymosin alpha 1 peptide derivative comprises a C-terminal bonded to a polyethylene glycol polymer at a first end of the polyethylene glycol polymer.

2. A pharmaceutical composition as in claim 1, wherein the polyethylene glycol polymer is bonded only at the first end of the polyethylene glycol polymer to the Thymosin alpha 1 peptide derivative.

3. A pharmaceutical composition as in claim 1, wherein the polyethylene glycol polymer defines a second end, and wherein the second end of the polyethylene glycol polymer is bonded to a second Thymosin alpha 1 peptide derivative.

4. A pharmaceutical composition as in claim 1, wherein the polyethylene glycol polymer has a molecular weight of about 5,000 to about 80,000.

5. A pharmaceutical composition as in claim 1, wherein the polyethylene glycol polymer has a molecular weight of about 8,000 to about 60,000.

6. A pharmaceutical composition as in claim 1, wherein the polyethylene glycol polymer has a molecular weight of about 10,000 to about 50,000.

7. A pharmaceutical composition as in claim 1, wherein the polyethylene glycol polymer has a molecular weight of about 20,000 to about 40,000.

8. A pharmaceutical composition as in claim 1, wherein the polyethylene glycol polymer comprises a straight-chain polymer.

9. A pharmaceutical composition as in claim 1, wherein the polyethylene glycol polymer comprises a branched-chain polymer.

10. A pharmaceutical composition as in claim 1, wherein the thymosin alpha 1 peptide derivative comprising a C-terminal bonded to the polyethylene glycol polymer comprises:
A-Ser-Asp-Ala-Ala-Val-Asp-Thr-Ser-Ser-Glu-Ile-Thr-
Thr-B-Asp-Leu-C-Glu-D-E-Glu-Val-Val-Glu-Glu-Ala-
Glu-Asn-X-Y-Z
wherein,
A represents H or Ac;
B,C,D,E represent Lys or Arg;
X is selected from the groups consisting of (Gly)n, (Gly-Ser)n, (Gly-Gly-Ser)n, and (Ser-Gly-Gly)n, wherein 1≦n≦10;
Y represents Cys, homo-Cys, Lys, Arg, or His, and wherein Y comprises the C-terminal bonded to the polyethylene glycol polymer; and
Z represents OH or NH₂.

11. A pharmaceutical composition as in claim 10, wherein
A is Ac;
B,C,D,E are each Lys;
X is Gly-Gly; and
Y is Cys bonded to the polyethylene glycol.

12. A pharmaceutical composition as in claim 10, wherein
A is Ac;
B,C,D,E are each Arg;
X is Gly-Gly; and
Y is Lys bonded to the polyethylene glycol.

13. A pharmaceutical composition as in claim 1, wherein the thymosin alpha 1 peptide derivative comprises a structure selected from the group consisting of SEQ ID NO: 1-24.

14. A pharmaceutical composition as in claim 1, wherein the thymosin alpha 1 peptide derivative comprises a structure selected from the group consisting of SEQ ID NO: 1-12.

15. A method for preparing a thymosin alpha 1 peptide derivative modified with polyethylene glycol, the method comprising
preparing the thymosin alpha 1 peptide derivative, wherein the thymosin alpha 1 derivative comprises a C-terminal; and
conjugating the C-terminal of the thymosin alpha 1 peptide derivative with a polyethylene glycol polymer.

16. A method as in claim 15, further comprising
preparing a polyethylene glycol containing a Michael Addition receptor and a thymosin alpha 1 peptide derivative containing a Michael Addition donator; and
reacting the polyethylene glycol containing the Michael Addition receptor with the thymosin alpha 1 peptide derivative containing the Michael Addition donator to form a thymosin alpha 1 peptide derivative modified with polyethylene glycol.

17. A method as in claim 15, further comprising
preparing a polyethylene glycol containing a Michael Addition donator and a thymosin alpha 1 peptide derivative containing a Michael Addition receptor; and
reacting the polyethylene glycol containing the Michael Addition donator with the thymosin alpha 1 peptide derivative containing the Michael Addition receptor to form the thymosin alpha 1 peptide derivative modified with polyethylene glycol.

18. A method as in claim 15, wherein the C-terminal of the thymosin alpha 1 peptide derivative comprises a cysteine, homocysteine, lysine or histidine.

19. A method as in claim 15, wherein the C-terminal of the thymosin alpha 1 peptide derivative is conjugated to the polyethylene glycol polymer via a covalent bond formed by chemical synthesis comprising at least one of the following processes:
(a) reacting the polyethylene glycol polymer with the Thymosin alpha 1 peptide derivative to generate an asymmetry disulfide bond forming the thymosin alpha 1 derivative conjugated to the polyethylene glycol polymer;
(b) reacting a polyethylene glycol polymer containing an activated carboxyl group with a thymosin alpha 1 peptide derivative having a C-terminal comprising lysine or histidine;
(c) performing a reductive amination reaction between a polyethylene glycol polymer containing a carbonyl group and a thymosin alpha 1 peptide derivative having a C-terminal comprising lysine;
(d) reacting a polyethylene glycol polymer containing an isocyano group or an isothiocyano group with a thymosin alpha 1 peptide derivative having a C-terminal comprising lysine;
(e) forming a urethane bond between a polyethylene glycol polymer containing an activated carbonyl group and a thymosin alpha 1 peptide derivative having a C-terminal comprising lysine or histidine;
(f) reacting a polyethylene glycol polymer containing a 2-carbonyl acetaldehyde group with a thymosin alpha 1 peptide derivative having a C-terminal comprising arginine; or
(g) performing a nucleophilic substitution reaction between a polyethylene glycol polymer comprising a substitution group and a thymosin alpha 1 peptide derivative comprising a sulphydryal group.

20. A method of treating diseases related to an immune system of a patient, the method comprising
administering a thymosin alpha 1 peptide derivative modified with a polyethylene glycol polymer to the patient, wherein the thymosin alpha 1 peptide derivative comprises a C-terminal bonded to a polyethylene glycol polymer.

21. A method as in claim 20, wherein the disease includes rheum, chronic hepatitis, lower immune function, tumor and virus infection.


SEQUENCE LISTING (24 sequences)

SEQ ID NO: 1 - protein, 30 aa, Artificial Sequence
  Description of Artificial Sequence Thymosin alpha 1 peptide derivative
SDAAVDTSSEITTKDLKEKKEVVEEAENGG

SEQ ID NO: 2 - protein, 30 aa, Artificial Sequence
  Description of Artificial Sequence Thymosin alpha 1 peptide derivative
SDAAVDTSSEITTRDLKEKKEVVEEAENGG

SEQ ID NO: 3 - protein, 30 aa, Artificial Sequence
  Description of Artificial Sequence Thymosin alpha 1 peptide derivative
SDAAVDTSSEITTKDLREKKEVVEEAENGG

SEQ ID NO: 4 - protein, 30 aa, Artificial Sequence
  Description of Artificial Sequence Thymosin alpha 1 peptide derivative
SDAAVDTSSEITTKDLKERKEVVEEAENGG

SEQ ID NO: 5 - protein, 30 aa, Artificial Sequence
  Description of Artificial Sequence Thymosin alpha 1 peptide derivative
SDAAVDTSSEITTKDLKEKREVVEEAENGG

SEQ ID NO: 6 - protein, 30 aa, Artificial Sequence
  Description of Artificial Sequence Thymosin alpha 1 peptide derivative
SDAAVDTSSEITTRDLREKKEVVEEAENGG

SEQ ID NO: 7 - protein, 30 aa, Artificial Sequence
  Description of Artificial Sequence Thymosin alpha 1 peptide derivative
SDAAVDTSSEITTRDLKERKEVVEEAENGG

SEQ ID NO: 8 - protein, 30 aa, Artificial Sequence
  Description of Artificial Sequence Thymosin alpha 1 peptide derivative
SDAAVDTSSEITTRDLKEKREVVEEAENGG

SEQ ID NO: 9 - protein, 30 aa, Artificial Sequence
  Description of Artificial Sequence Thymosin alpha 1 peptide derivative
SDAAVDTSSEITTKDLRERKEVVEEAENGG

SEQ ID NO: 10 - protein, 30 aa, Artificial Sequence
  Description of Artificial Sequence Thymosin alpha 1 peptide derivative
SDAAVDTSSEITTKDLREKREVVEEAENGG

SEQ ID NO: 11 - protein, 30 aa, Artificial Sequence
  Description of Artificial Sequence Thymosin alpha 1 peptide derivative
SDAAVDTSSEITTKDLKERREVVEEAENGG

SEQ ID NO: 12 - protein, 30 aa, Artificial Sequence
  Description of Artificial Sequence Thymosin alpha 1 peptide derivative
SDAAVDTSSEITTRDLRERREVVEEAENGG

SEQ ID NO: 13 - protein, 30 aa, Artificial Sequence; identical to SEQ ID NO: 11
  Description of Artificial Sequence Thymosin alpha 1 peptide derivative

SEQ ID NO: 14 - protein, 30 aa, Artificial Sequence; identical to SEQ ID NO: 10
  Description of Artificial Sequence Thymosin alpha 1 peptide derivative

SEQ ID NO: 15 - protein, 30 aa, Artificial Sequence; identical to SEQ ID NO: 10
  Description of Artificial Sequence Thymosin alpha 1 peptide derivative

SEQ ID NO: 16 - protein, 30 aa, Artificial Sequence; identical to SEQ ID NO: 9
  Description of Artificial Sequence Thymosin alpha 1 peptide derivative

SEQ ID NO: 17 - protein, 30 aa, Artificial Sequence; identical to SEQ ID NO: 8
  Description of Artificial Sequence Thymosin alpha 1 peptide derivative

SEQ ID NO: 18 - protein, 30 aa, Artificial Sequence; identical to SEQ ID NO: 7
  Description of Artificial Sequence Thymosin alpha 1 peptide derivative

SEQ ID NO: 19 - protein, 30 aa, Artificial Sequence; identical to SEQ ID NO: 6
  Description of Artificial Sequence Thymosin alpha 1 peptide derivative

SEQ ID NO: 20 - protein, 30 aa, Artificial Sequence
  Description of Artificial Sequence Thymosin alpha 1 peptide derivative
SDAAVDTSSEITTRDLRERKEVVEEAENGG

SEQ ID NO: 21 - protein, 30 aa, Artificial Sequence
  Description of Artificial Sequence Thymosin alpha 1 peptide derivative
SDAAVDTSSEITTRDLREKREVVEEAENGG

SEQ ID NO: 22 - protein, 30 aa, Artificial Sequence
  Description of Artificial Sequence Thymosin alpha 1 peptide derivative
SDAAVDTSSEITTKDLRERREVVEEAENGG

SEQ ID NO: 23 - protein, 30 aa, Artificial Sequence
  Description of Artificial Sequence Thymosin alpha 1 peptide derivative
SDAAVDTSSEITTRDLKERREVVEEAENGG

SEQ ID NO: 24 - protein, 30 aa, Artificial Sequence; identical to SEQ ID NO: 12
  Description of Artificial Sequence Thymosin alpha 1 peptide derivative